(19) United States
(12) Patent Application Publication
(10) Pub. No.: US 2022/0000804 A1
(43) Pub. Date: Jan. 6, 2022

(54) INTRANASAL NANO INDUCER FOR PREVENTING AND TREATING NEURODEGENERATIVE DISEASES AND METHOD THEREOF

(71) Applicant: Nasal-Phyto (SZ) Pharmaceutical Technology Co., Ltd., Shenzhen (CN)

(72) Inventors: Gang LIU, Shenzhen (CN); Jingyi LIU, Shenzhen (CN); Lei WEN, Shenzhen (CN); Jinfeng ZHANG, Shenzhen (CN)

(21) Appl. No.: 17/372,403

(22) Filed: Jul. 9, 2021

Related U.S. Application Data
(63) Continuation of application No. PCT/CN2020/070987, filed on Jan. 8, 2020.

(30) Foreign Application Priority Data
Jan. 9, 2019 (CN) 201910019951.6

(51) Int. Cl. A61K 31/12; A61K 47/36; A61K 47/10; A61P 25/16

(52) U.S. Cl. CPC A61K 31/12; A61P 25/16; A61K 47/10; A61K 47/36

(57) ABSTRACT

An intranasal nano autophagy inducer for preventing and treating early neurodegenerative diseases comprises hydrophobic molecules (i.e., hydrophobic small molecules or autophagy inducer) having autophagy inducing effects and amphiphilic surfactant. Firstly configuring a good solvent solution, preparing a suspension emulsion of self-contained nanoparticles without carriers by a reprecipitation method, freeze-drying to prepare a freeze-dried powder, and resuspending the freeze-dried powder in isotonic saline to obtain the intranasal nano autophagy inducer before immediate use.

### RELATED APPLICATIONS

This application is a continuation of International Patent Application PCT/CN2020/070987, filed on Jan. 8, 2020, which claims priority to Chinese patent application number 201910019951.6, filed on Jan. 9, 2019. International Patent Application PCT/CN2020/070987 and Chinese patent application number 201910019951.6 are incorporated herein by reference.

### FIELD OF THE DISCLOSURE

The present disclosure relates to the field of biopharmaceuticals, and relates to an intranasal nano inducer and a method thereof. Specifically, the present disclosure relates to an intranasal nano inducer for preventing and treating neurodegenerative diseases and a method thereof.

### BACKGROUND OF THE DISCLOSURE

Autophagy is a process of engulfing a body's own cytoplasmic proteins or organelles, engulfing the cytoplasmic proteins or organelles into vesicles, fusing with lysosomes, and degrading the contents of the cells, thereby fulfilling metabolic needs and updating of organelles. Neurons are severely dependent on autophagy. The destruction of the autophagy pathway can lead to accumulation of ubiquitinated protein aggregates in neurons, induce neuronal degeneration, and then lead to the occurrence of neurodegenerative diseases.

Alzheimer's disease (AD) is the most common neurodegenerative disease. Its two main characteristics are the formation of senile plaques caused by the accumulation of amyloid β peptide and the formation of neurofibrillary tangles caused by the accumulation of Tau. The autophagy pathway is activated and effectively degraded. The prevalence of Parkinson's disease (PD) in people over 60 years old is 1%, mainly manifested as motor dysfunction such as tremors, which are caused by the degeneration of dopaminergic neurons. Its pathogenesis is directly related to defects in the autophagy-lysosomal pathway, and the main pathological marker is the abnormal aggregate Lewy body of α-synuclin.

According to multiple clinical reports, olfactory dysfunction is a common symptom in the early stages of neurodegenerative diseases such as AD and PD. More than 90% of Parkinson's patients have olfactory dysfunction, which can be used as an indicator to distinguish PD from atypical Parkinson's syndrome and provides help for the early diagnosis and differential diagnosis of PD. The olfactory mechanism of neurodegenerative diseases is related to the accumulation of toxic proteins in the olfactory area. Under pathological conditions, the autophagy pathway in the olfactory-related area is damaged, causing the protein to fail to be degraded normally and accumulating and compressing neurons, which induces the progression of the disease. For these abnormal protein aggregations, there is currently no clinically effective medicine. For example, the treatment of PD can only relieve symptoms by supplementing dopamine and cannot treat the disease. And as the disease worsens, dopamine therapy gradually fails, and serious side effects occur at the same time. Therefore, there is an urgent need to develop new therapeutic drugs.

Autophagy inducers can enhance the occurrence of autophagy flux in neurons through a variety of mechanisms, such as increasing the production of autophagosomes, promoting the fusion of autophagosomes and lysosomes, enhancing lysosome function, increasing the number of lysosomes, and so on, repairing the damaged autophagy pathway, promoting the degradation of toxic proteins, and reducing the nerve damage caused by its accumulation. Certain autophagy inducers can activate the autophagy pathway by binding to Transcription factor EB (TFEB) protein and are effective autophagy inducers. However, because autophagy inducers are a small organic molecule with strong hydrophobicity and poor druggability, autophagy inducers have low bioavailability, less accumulation in the brain, and short circulation time in the body when administered orally. Various problems limit autophagy inducers exploratory research on the AD, PD, other pathology models, and its application of future clinical transformation.

With the rapid development of nanotechnology, nano-preparations have been widely used in the fields of medicine and biology because of their advantages, such as protecting drugs from being destroyed, prolonging effective drug maintenance time, controlling drug release, and reducing drug side effects. It has been reported that there are many nano-drug particles encapsulated by liposomes, polymers, and other carriers, usually with a drug load of less than 10%, and the accumulation of polymers and other carriers in the brain may potentially be toxic, bring side effects, and become a hindrance to nanotechnology granule for further clinical application. In addition, the blood-brain barrier, as an important physiological barrier, prevents more than 98% of drugs from entering the brain tissue, especially nano-drugs with a diameter of about 100 nm, making it more difficult to penetrate the blood-brain barrier to reach the nervous system and play a role. Therefore, there is an urgent need to develop a nano-delivery system with high drug loading, high brain targeting, and safety and non-toxicity for the treatment of neurological diseases by autophagy small molecule drugs.

### BRIEF SUMMARY OF THE DISCLOSURE

The present disclosure provides an intranasal nano autophagy inducer (i.e., nano inducer) and a method thereof. In detail, the present disclosure provides a self-contained intranasal nano autophagy inducer without carriers and a method thereof to prevent and treat early neurodegenerative diseases (e.g., neurodegenerative diseases).

A technical solution of the present disclosure is as follows.

An intranasal nano autophagy inducer for preventing and treating early neurodegenerative diseases comprises hydrophobic molecules (e.g., hydrophobic small molecules) having autophagy inducing effects and amphiphilic surfactant. Firstly configuring a good solvent solution having 1-10 mg/mL of amphiphilic surfactant and 0.5-5 mg/mL of the hydrophobic molecules having autophagy inducing effects, then dripping the good solvent solution into deionized water, wherein a volume ratio of the good solvent solution (e.g., solvent solution) and the deionized water is (0.5-5):50, blowing gas during the dripping to assist in a volatilization of a solvent of the good solution, preparing a suspension emulsion of self-contained nanoparticles without carriers with a particle size of 50-200 nm by a reprecipitation method, freeze-drying to prepare a freeze-dried powder, and resuspending the freeze-dried powder in isotonic saline to obtain the intranasal nano autophagy inducer before use (i.e., immediate use).

The autophagy inducer of the present disclosure can induce autophagy and eliminate abnormal protein accumulations.

Preferably, a surface potential of the self-contained nanoparticles without carriers is −10 to −60 mV. More preferably −10 to −30 mV.

Preferably, the early neurodegenerative diseases comprises Alzheimer's disease and Parkinson's disease.

The early neurodegenerative diseases are accompanied with olfactory disorder symptoms, and the autophagy inducer is an intranasal nano autophagy inducer that is highly targeted and enriched in olfactory bulb. The intranasal nano autophagy inducer of the present disclosure significantly eliminate the abnormal protein accumulations in olfactory area and other pathological change areas.

Preferably, the hydrophobic small molecule is a curcumin analog having the following structural formula, cis-isomers of the curcumin analog, or a mixture of the cis-isomers of the curcumin analog and the curcumin analog in any ratio:

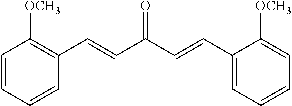 [SMILES: COc1ccccc1/C=C/C(=O)/C=C/c1ccccc1OC]

Preferably, the hydrophobic small molecule the mixture of the cis-isomers of the curcumin analog and the curcumin analog. A weight ratio of the cis-isomers is 25-35% of a total mixture in the mixture.

Preferably, the mixture in which a weight ratio of the cis-isomers is 25-35 wt % of a total mixture is prepared by the following method. A methanol solution of the curcumin analog is irradiated by ultraviolet (UV) for 1.5-2.5 hours.

Preferably, a concentration of the methanol solution of the curcumin analog is 0.5-5 mg/mL, more preferably 0.5-1.5 mg/mL. When a time for UV irradiation is less than 1.5 hours, no enough amount of the cis-isomers are generated, and when the time for UV irradiation is more than 2.5 hours, by-products are generated. Even more preferably, the time for UV irradiation is 2 hours.

Preferably, the autophagy inducer further comprises chitooligosaccharides, wherein a concentration of the chitooligosaccharides in the isotonic saline solution is 0.01-0.2% (w/v).

In the present disclosure, amphiphilic surfactant is not limited can be pharmaceutically acceptable, have a lipophilic group and a hydrophilic group, and be configured to be in combination with the autophagy inducer (e.g., autophagy inducing drug molecules) to form self-assembled nanoparticles. Preferably, the amphiphilic surfactant of the present disclosure is a polyethylene glycol derivative. More preferably, the polyethylene glycol derivative is a polyethylene glycol derivative having a carboxyl group or polymaleic anhydride 18 carbene-polyethylene glycol.

Preferably, the gas is nitrogen gas or inert gas. Preferably, nitrogen gas. Gas blowing assists a volatilization of the good solvent to ensure a generation of nanoparticles and to prevent safety threats caused by solvent residues.

Preferably, an average particle size of the intranasal nano autophagy inducer is 50-200 nm, preferably 50-150 nm, more preferably 50-120 nm.

Preferably, a drug load rate of the intranasal nano autophagy inducer is more than 25%.

Preferably, the intranasal nano autophagy inducer of the present disclosure further comprises chitooligosaccharides. The concentration of chitooligosaccharides in an isotonic saline solution is 0.01-0.2% (w/v). When in use, the freeze-dried powder is resuspended in the isotonic saline containing chitooligosaccharides.

Chitooligosaccharide, also named as oligochitosan and oligochitosan, has a molecular weight of ≤3200 Da and has many unique functions such as higher solubility, fully soluble in water, and is easy to be absorbed by organisms than chitosan. Chitooligosaccharide is non-toxic functional low-molecular-weight amino sugar with polycationic structures. The present disclosure enables the chitooligosaccharides to be modified on outer sides of the nanoparticles to prevent the drug from stimulating an environment in a nasal cavity. The chitooligosaccharide is easy to interact with negatively charged groups on surfaces of mucosal cells to change fluidity and permeability of cell membranes, and increase absorption of the nanoparticles. In addition, the chitooligosaccharide itself further has a certain immunomodulatory and neuroprotective effect, and effects of the chitooligosaccharide is 14 times of that of chitosan.

A degree of polymerization of the chitooligosaccharide used in the present disclosure is 2-20, or a molecular weight of the chitooligosaccharide is ≤3200 Da.

Preferably, the concentration of the oligosaccharide of the present disclosure in the isotonic saline solution is 0.01-0.2% (w/v). When the concentration is less than 0.01%, it is difficult to increase absorption and prevent from irritation. When the concentration is greater than 0.2%, it is easy to cause the negatively charged nanoparticles to accumulate.

The freeze-dried powder is resuspended in isotonic saline, and a concentration of the freeze-dried powder can be configured as required. Preferably, 3-7 mg/mL.

A formation of nanoparticles is affected by the molecular structure of hydrophobic small molecules, and there is a non-covalent bonding force between the molecules, which can lead to nanostructure differences due to different molecular configurations. In order to enhance a stability of the nanoparticles, before a solvent exchange in the present disclosure, an amphiphilic surfactant is added and well mixed with the hydrophobic small molecules in a certain ratio in an organic solvent, the solvent exchange is then performed, and the nanoparticles without carriers are obtained by a reprecipitation method.

The intranasal nano autophagy inducer of the present disclosure has no other carrier components, so it has high drug load amount, low toxicity, good safety, small and uniform particle size, high stability, and long circulation time in vivo.

In the present disclosure, a drug contained in the intranasal nano autophagy inducer is a small hydrophobic organic molecule with an autophagy-inducing effect. After its druggability is improved, it can be used to prevent and treat neurodegenerative diseases.

The present disclosure can further be added with antioxidants, the antioxidants can be one or more of sodium metabisulfite, sodium bisulfite, sodium sulfite, sodium thiosulfate, cysteine hydrochloride, vitamin C, vitamin E, or sulfur urea, and its dosage is a pharmaceutically prescribed conventional dosage.

The present disclosure can further be added with preservatives, and the preservatives can be one or more of methyl paraben, ethyl paraben, propyl paraben, butyl paraben, Benzalkonium bromide, benzalkonium chloride, chlorobutanol, phenethyl alcohol, thimerosal, phenylmercury nitrate, sorbic acid, or chlorhexidine, and its dosage is a pharmaceutically prescribed conventional dosage.

The present disclosure can further be added with an osmotic pressure regulator, and the osmotic pressure regulator can be one or more of sodium chloride, glucose, lactose, or mannitol, and a dosage is a pharmaceutically prescribed conventional dosage.

The present disclosure provides a self-contained intranasal nano autophagy inducer without carriers. The nano particles are spherical or approximately spherical, and the surface potential is negative. The hydrophobic autophagy-inducing drug molecules are prepared into nanoparticle suspension emulsion by the reprecipitation method and are freeze-dried to obtain nanoparticle freeze-dried powder. The freeze-dried powder is resuspended in isotonic saline to obtain the intranasal nano autophagy inducer before immediate use, which is administered using nasal drops or nasal spray for a treatment of neurodegenerative diseases.

The present disclosure provides the intranasal nano autophagy inducer for preventing and treating early neurodegenerative diseases, a delivery drug is a hydrophobic small molecule with autophagy inducing effect, and a delivery method is a self-contained intranasal nano delivery system without carriers using nose drops or spray method, it is non-invasive, no carriers, no biodegradation problems, and accumulated toxicity, the drug load rate is up to 25% or more, it highly retains the binding ability of small molecules and target receptors and can be pH-responsive and slow-released molecular drugs in neurons, and the autophagy pathway is specifically activated. Therefore, the accumulation of toxic proteins in the brain of neurodegenerative diseases is efficiently eliminated, especially for olfactory disorders in an early stage, and it has great significance for preventing further deterioration of the disease.

In the second aspect, the present disclosure provides a method for preparing the intranasal nano autophagy inducer as described in the first aspect, the method comprising the following steps:

1) configuring a good solvent solution having 1-10 mg/mL of amphiphilic surfactant and 0.5-5 mg/mL of the hydrophobic molecules having autophagy inducing effects, then dripping the good solvent solution into deionized water, a volume ratio of the good solvent solution and the deionized water is (0.5-5):50, blowing gas while the dripping to assist a volatilization of the good solvent;

2) Preparing a suspension emulsion of self-contained nanoparticles without carriers with a particle size of 50-200 nm by a reprecipitation method, freeze-drying to prepare a freeze-dried powder; and

3) Resuspending the freeze-dried powder in isotonic saline to obtain the intranasal nano autophagy inducer before immediate use.

The method for preparing the intranasal nano autophagy inducer of the present disclosure is prepared by a reprecipitation method. When the good solvent is converted into water (poor solvent), the hydrophobic small molecules with autophagy inducing effect are precipitated to form nanoparticles, and the amphiphilic surfactant is added to further enhance its stability and water dispersibility. The method is simple is easy to be performed, does not require complicated operations and conditions, and can be performed at room temperature.

It should be emphasized that in the present disclosure, the amphiphilic surfactant should be added before a formation of nanoparticles, that is, dissolving amphiphilic surfactant and small molecules together in the good solvent, uniformly mixing, then dripping into the water phase to prepare nano-particles, and blowing gas (preferably nitrogen) while dripping to remove organic solvent. This method is different from a method in which nanoparticles are firstly formed and then amphiphilic surfactant is added for surface modification, and products are also different.

In the method for preparing the intranasal nano autophagy inducer of the present disclosure, the good solvent is mutually soluble with water. According to Flory-Krigboum dilute solution theory, the good solvent refers to a solvent whose interaction parameter with a solute is less than 0.5.

Preferably, the good solvent is one or a mixture of acetone, methanol, ethanol, or tetrahydrofuran, more preferably tetrahydrofuran.

In the method for preparing the intranasal nano autophagy inducer of the present disclosure, the water can be deionized water, distilled water, double distilled water, etc., preferably deionized water.

In the method for preparing the intranasal nano autophagy inducer of the present disclosure, a concentration of the hydrophobic molecules (i.e., hydrophobic drug molecules) dissolved in the good solvent in the step (1) is 0.5-5 mg/mL, preferably 1 mg/mL.

In the method for preparing the intranasal nano autophagy inducer of the present disclosure, a volume ratio of the good solvent and water in the step (1) is preferably (1-3):50, such as 1:50, 1.2:50, 1.5:50, 1.8:50, 1.9:50, 2.1:50, 2.5:50, or 2.8:50, preferably 2:50.

Preferably, a reaction temperature in the step (1) is 20-30° C., more preferably 25° C.

In the method for preparing the intranasal nano autophagy inducer of the present disclosure, a concentration of the amphiphilic surfactant added in the step (2) in the good solvent can be 1-10 mg/mL, for example, 1 mg/mL, 2 mg/mL, 3 mg/mL, 4 mg/mL, 5 mg/mL, 6 mg/mL, 7 mg/mL, 8 mg/mL, 9 mg/mL, or 10mg/mL, preferably 2 mg/mL.

Preferably, an ultrasonic dispersion time in the step (2) is 3-30 minutes, such as 3 minutes, 4 minutes, 5 minutes, 8 minutes, 10 minutes, 15 minutes, 20 minutes, 25 minutes, or 28 minutes, more preferably 5 minutes.

Self-assembled nanoparticles may accumulate in aqueous solution after long-term storage. The self-assembled nanoparticles are Freeze-dried to significantly improve stabilities of the self-assembled nanoparticles. A freeze-drying temperature is lower than a eutectic point at which nanoparticles and water coexist by 10-20° C., and the self-assembled nanoparticles are freeze-dried under 10 Pa for 24-90 hours, preferably 48 hours.

In order to avoid nanoparticle accumulation and particle size changes after freeze-drying, cryoprotectants, such as glucose, mannitol, lactose, NaCl, etc. are firstly added to promote a formation of a large number of tiny ice crystals during a freeze-drying process or to enable the freeze-dried product to define in a loose state. The nanoparticles retain original forms and are easily redispersed in water.

The intranasal nano autophagy inducer of the present invention can be administered through the nasal cavity in the form of spray or nasal drops.

Compared with the existing techniques, the technical solution has the following advantages.

(1) The present disclosure relates to an intranasal nano autophagy inducer. The drug can be absorbed through the mucosa of the olfactory area under the route of intranasal administration, and firstly reach the olfactory-related area of the brain and be enriched there. It has a specific alleviating effect for the symptoms common to early neurodegenerative diseases, it has a significant effect of clearing abnormal protein accumulation in the olfactory area and other diseased areas, and it is of great significance to prevent the further deterioration of early AD, PD, and other neurodegenerative diseases.

(2) The present disclosure relates to a high drug loading, high brain targeting, safe, and non-toxic intranasal nano autophagy inducer delivery system, which is applied to hydrophobic organic small molecules with autophagy inducing effect and is simple to operate, has wide application range, and has strong universality. Compared with prodrugs with small molecules, it has the advantages of significantly improving water dispersibility and druggability, enhancing bioavailability, reducing the frequency of administration, reducing toxicity, and reducing side effects. Compared with the traditional liposome or polymer nano drug delivery system, this nano system has no carrier, no biodegradation problem and accumulated toxicity, the drug loading rate is as high as 25% or more, and the target receptor binding ability of the original molecule is highly retained. It has pH-responsive drug release characteristics, which can play a role in being long-acting and having a slow-release, and it has a good application prospect in the treatment of neurological diseases.

(3) The intranasal nano autophagy inducer in the present disclosure is provided through the nasal brain direct pathway, bypassing the blood-brain barrier along the olfactory nerve and other pathways, to efficiently deliver the drug into the brain, thereby avoiding the degradation in the gastrointestinal tract and the first-pass effect of the liver, therefore enhancing brain targeting. It has the characteristics of high bioavailability, fast onset, and good patient compliance, and it can reduce the accumulation in organs of the peripheral circulatory system and reduce the potential side effects due to long-term use. Compared with oral drugs, nasal preparations have no first-pass effect and reduce drug consumption. Compared with intravenous injection, nasal administration only requires nasal drops, sprays, etc., which is convenient and non-invasive, and improves patient compliance. Especially for patients with neurodegenerative diseases who take drugs for a long time, it can alleviate the pain of the patient, has good compliance with the patient, facilitates self-administration, reduces the risks caused by long-term medication, and has a good application prospect.

### BRIEF DESCRIPTION OF THE DRAWINGS

The present disclosure will be further described below in combination with the accompanying drawings and embodiments.

FIGS. 1a-1b illustrate scanning electron micrographs (SEMs) of intranasal nano autophagy inducers. FIG. 1a illustrates M1 nanoparticles prepared in Embodiment 1, and FIG. 1b illustrates M1 nanoparticles carrying TPAAQ probes prepared in Embodiment 2.

FIGS. 2a-2b illustrate transmission electron micrographs (TEMs) of the intranasal nano autophagy inducers. FIG. 2a illustrates the M1 nanoparticles prepared in Embodiment 1, and FIG. 2b illustrates the M1 nanoparticles carrying the TPAAQ probes prepared in Embodiment 2.

FIGS. 3a-3b illustrate particle size distribution graphs of the intranasal nano autophagy inducers. FIG. 3a illustrates the M1 nanoparticles prepared in Embodiment 1, and FIG. 3b illustrates the M1 nanoparticles carrying the TPAAQ probes prepared in Embodiment 2.

FIGS. 4a-4b illustrate a drug load rate of the intranasal nano autophagy inducers. FIG. 4a illustrates the M1 nanoparticles prepared in Embodiment 1, and FIG. 4b illustrates the M1 nanoparticles carrying the TPAAQ probes prepared in Embodiment 2.

FIG. 4c illustrates a standard curve of the M1 nanoparticles prepared in Embodiment 1.

FIG. 4d illustrates an absorption spectrum of M1 nanoparticles prepared in Embodiment 1.

FIG. 5 illustrates a surface potential graph of the M1 nanoparticles prepared in Embodiment 1.

FIG. 6 illustrates an optical characterization graph with respect to Tyndall effect of the M1 nanoparticles prepared in Embodiment 1.

FIG. 7 illustrates a pH-responsive drug release curve of the M1 nanoparticles prepared in Embodiment 1.

FIG. 8 illustrates a cytotoxicity graph of the M1 nanoparticles prepared in Embodiment 1 and a small molecule M1 drug.

FIG. 9 illustrates neuroprotective effects in vitro of the M1 nanoparticles prepared in Embodiment 1.

FIG. 10 illustrates a validation graph of a combination effect of the M1 nanoparticles prepared in Embodiment 1 and a TFEB protein of a target protein.

FIG. 11 illustrates a cell autophagic flux fluorescence graph induced by the M1 nanoparticles prepared in Embodiment 1.

FIG. 12 illustrates a cell uptake of the M1 nanoparticles carrying the TPAAQ probes (i.e., TPAAQ fluorescent probes) prepared in Embodiment 2.

FIG. 13 illustrates a M1 drug content graph in brain and plasma of mice of M1 nanoparticles prepared in Embodiment 3 after nasal and brain administration.

FIG. 14 illustrates a fluorescence biodistribution graph of brain and organs of mice of M1 nanoparticles carrying TPAAQ fluorescent probes prepared in Embodiment 4 after nasal administration.

FIG. 15a-d illustrate an open field behavior graph of Parkinson's model mice in Embodiment 5.

FIG. 16 illustrates a gait behavior graph of the Parkinson's model mice in Embodiment 5.

FIG. 17 illustrates a distribution graph of olfactory bulb, striatum, and substantia nigra of the Parkinson's model mice in Embodiment 5 after nasal administration of M1 nanoparticles, where an arrow shows the M1 nanoparticles.

FIGS. 18a-18d illustrate an expression content test graph of toxic protein and autophagy pathway related protein of brain olfactory bulb of the Parkinson's model mice in Embodiment 5.

FIG. 19a-19c illustrate an expression content test graph of toxic protein and autophagy pathway related protein of brain substantia nigra of the Parkinson's model mice in Embodiment 5.

FIG. 20 illustrates a spectrum of a compound with a molecular weight of 294.34 prepared in Embodiment 1.

FIG. 21a-21g illustrate a conversion rate of curcumin analog in Embodiment 1 under different conditions.

FIG. 22 illustrates a nuclear magnetic spectrum of a compound having a (e, e) double trans configuration.

FIG. 23 illustrates a nuclear magnetic spectrum of a compound having (e, z) cis-trans configuration.

FIG. 24 illustrates a high-performance liquid chromatography mass spectrometry (HPLC-MS) spectrum of a mixture.

### DETAILED DESCRIPTION OF THE EMBODIMENTS

The embodiments of the present disclosure will be further described below in combination with the accompanying embodiments. Those skilled in the art should understand that the aforementioned embodiments are merely some preferred embodiments of the present disclosure to better understand the present disclosure, and the scope of the disclosure is not limited thereto. For those skilled in the art, the present disclosure can have various modifications and variations. It is intended that the present disclosure cover any modifications, equivalences, improvements, etc. of the presently presented embodiments provided they are made without departing from the spirit and the principle of the present disclosure. The experimental methods described in the following embodiments, unless otherwise specified, are conventional methods. The used experimental materials, unless otherwise specified, are purchased from conventional biochemical reagent manufacturers.

### Embodiment 1: Preparation of Mixed Isomers M1

The mixed isomers are a mixture of isomers of curcumin analog having the following structural formula:

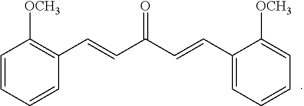 [SMILES: COc1ccccc1/C=C/C(=O)/C=C/c1ccccc1OC]

According to computer simulation results, in the mixture, when a ratio of a cis-isomer is higher, a biological activity of a product is stronger. While in practice, when a yield of the product is higher, a yield of by-products is also higher.

This embodiment provides a method for preparing an isomer product with a conversion rate of 30%. The preparation method is simple, and no by-products are generated.

Based on hydrophobic characters of curcumin analog, when the curcumin analog is dissolved in a good solvent and given different radiation conditions, isomer conversions will occur in different degrees, and a mixture of curcumin analog cis-trans isomers having different ratios can be obtained. Among them, a conversion rate under sunlight is the highest, but by-products are generated. A conversion rate under ultraviolet (UV) irradiation follows, and a conversion rate under radioactive iodine radiation follows. Temperature has no influence on structures of the curcumin analog under dark conditions.

Further, the good solvent is preferably acetonitrile, methanol, ethanol, acetone, or tetrahydrofuran.

Further, the isomer product with the conversion rate of 30% is obtained under UV irradiation for 2 hours. The preparation method is simple, and no by-products are generated.

### Method for Preparing a Mixture of the Curcumin Analog Cis-Trans Isomers M1 (Mixture 1)

A methanol solution containing 1 mg/mL of the curcumin analog is configured, sealed with aluminum foil, and respectively placed at 4° C., 25° C., or 50° C. for 8 hours to obtain reaction products 1-3. Another methanol solution containing the same concentration of the curcumin analog is configured, respectively exposed under sunlight for 2 hours, exposed under sunlight for 24 hours, exposed under ultraviolet for 2 hours, or radiated under radioactive iodine 131 for 2 hours at room temperature (i.e., 20-22° C.) to obtain reaction products 4-7. The mixture of curcumin analog cis-trans isomers having different ratios is obtained.

### Results

### (1) Molecular Weight Identification of the Conversion Product

A molecular weight of the conversion product is identified by high performance liquid chromatography (HPLC) in combination with time-of-flight mass spectrometer (TOF-MS). Referring to FIG. 20, molecular weights of the curcumin analog and the conversion product of the curcumin analog are both 294.34. Therefore, the two (e.g., the conversion product of the curcumin analog) are confirmed as isomers, and according to structures, the two are cis-trans isomers.

### (2) Evaluation of the Conversion Rates of the Cis-Trans Isomers

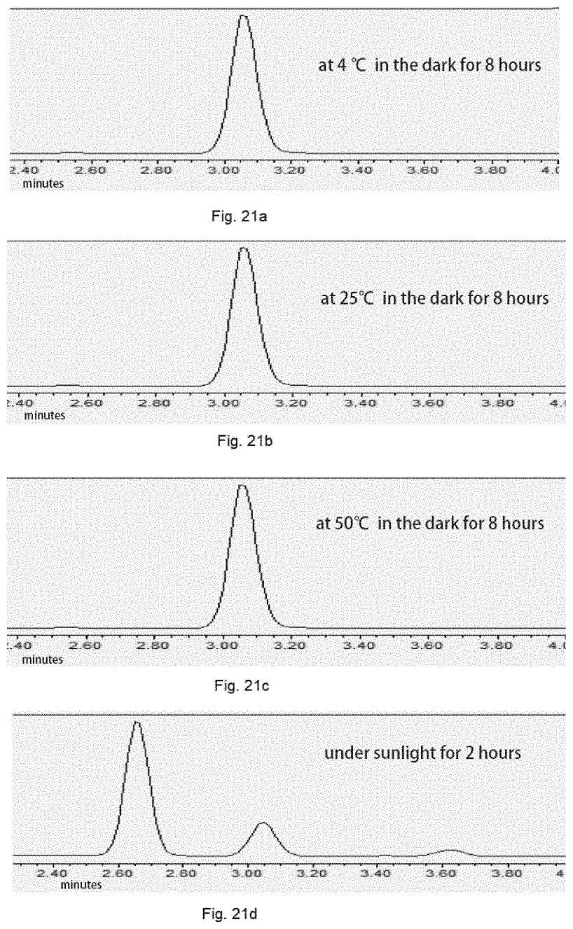

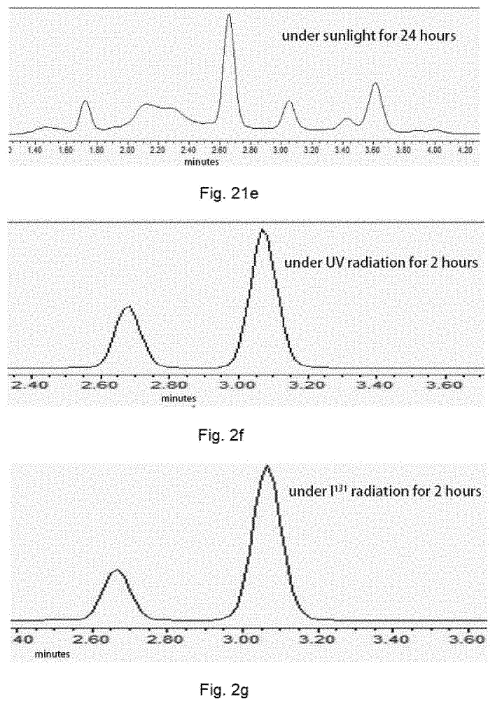

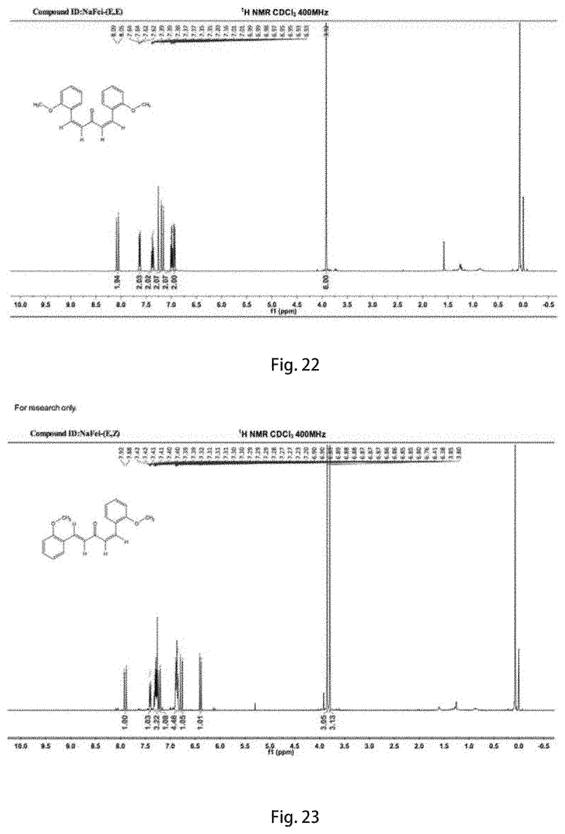

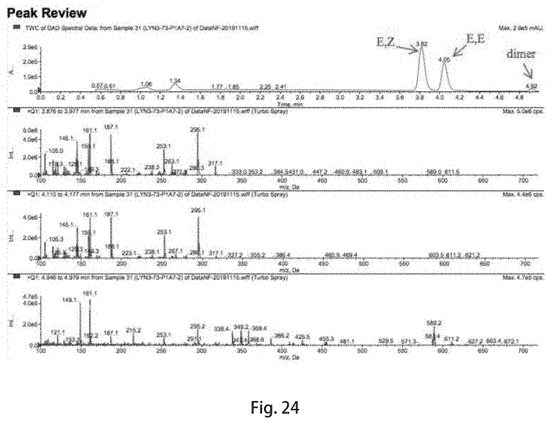

The conversion rates of the cis-trans isomers of the curcumin analog in sample solutions 1-7 (e.g., the reaction products 1-7) are evaluated by HPLC at a maximum absorption wavelength of 384 nm. Referring to FIG. 21, as a result, no new substances are generated in the reaction products 1-3, which indicates that the isomer conversions of the curcumin analog do not occur (FIGS. 21a-21c). After the reaction product 4 is exposed under sunlight for 2 hours, 73.91% content of the curcumin analog is converted to isomers of the curcumin analog (FIG. 21d). After the reaction product 5 is exposed under sunlight for 24 hours, many complex products are generated in addition to the isomers of the curcumin analog (FIG. 21e). After the reaction product 6 is exposed under UV irradiation for 2 hours, a conversion rate of the isomers of the curcumin analog is 29.59% (FIG. 21f). After the reaction product 7 is exposed under radioactive iodine 131 irradiation conditions for 2 hours, a conversion rate of the isomers of the curcumin analog is 27.91% (FIG. 21g).

According to the test results of Embodiment 1, the reaction product 6 is selected as representative mixed isomers M1 for subsequent biological activity research. The preparation method is simple and is easily controlled, and no by-products are generated (the conversion rate of the isomers of the curcumin analog under the UV irradiation for 2 hours is about 30%).

### Embodiment 2: preparation of an Intranasal Nano-Formulation of the Curcumin Analog M1 (e.g., Nanoparticles of the Curcumin Analog M1)

The curcumin analog M1 used in this embodiment is the reaction product 6 prepared in Embodiment 1.

5 mL of a tetrahydrofuran solution containing 1 mg/mL of the curcumin analog M1 and 2 mg/mL of carboxypolyethylene glycol is configured and mixed well, 200 μL of the solution of the curcumin analog M1 is taken and added dropwise into 5 mL of deionized water, nitrogen gas is blown while dripping to remove organic solvents, stirring at 25° C. for 10 minutes, and then still standing to obtain a suspension emulsion of self-contained nanoparticles of the curcumin analog M1 without carriers, then freeze-drying to form freeze-dried powders. Before immediate use, the freeze-dried powders are redispersed in isotonic saline, added dropwise to a chitooligosaccharide saline solution (0.1 weight/volume (w/v)), and stirred for 0.5-2 hours to obtain the self-contained intranasal nano-formulation of the curcumin analog M1 without carriers.

### Results

### (1) Evaluation of Morphology, Particle Sizes, and Potential Distribution of the Nanoparticles of the Curcumin Analog M1

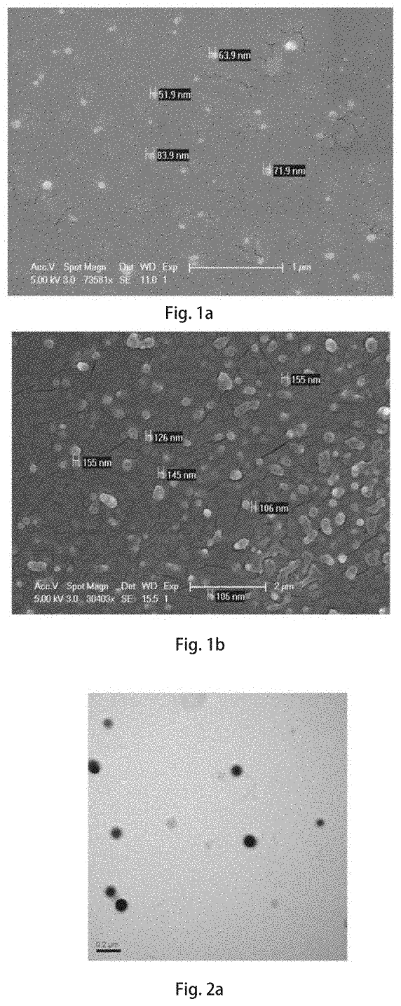

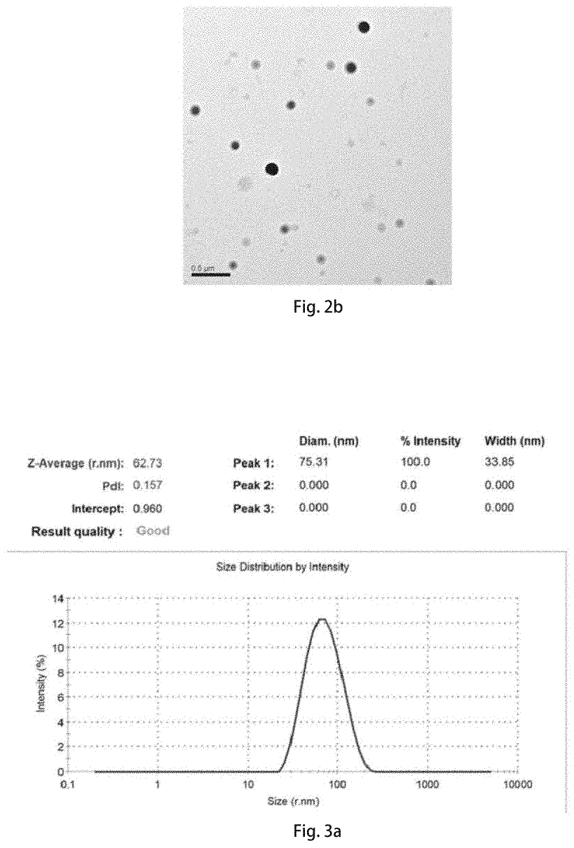

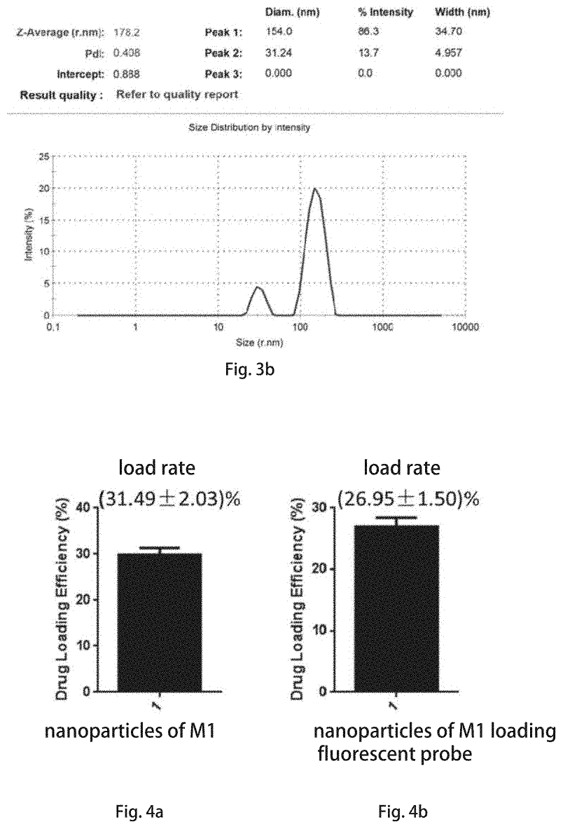

A scanning electron microscope (FEI Quanta200, Netherlands) is used to observe the nanoparticles of the curcumin analog M1 prepared in Embodiment 2 according to a method described in a specification of the scanning electron microscope. FIG. 1a illustrates a scanning electron micrograph. A high-resolution transmission electron microscope (FEI Technai F30, Netherlands) is used to observe the nanoparticles of the curcumin analog M1 prepared in Embodiment 2 according to a method described in a specification of the high-resolution transmission electron microscope. FIG. 2a illustrates a transmission electron micrograph. A laser particle size analyzer (Malvern, UK) is used to evaluate dynamic light scattering of the nanoparticles of the curcumin analog M1 prepared in Embodiment 2 according to a method described in a specification of the laser particle size analyzer. An average particle size of the nanoparticles of the curcumin analog M1 prepared in Embodiment 2 is evaluated to be 62.73 nm, and FIG. 3a illustrates a particle size distribution.

### (2) Evaluation of a Drug Load Rate of the Nanoparticles of the Curcumin Analog M1

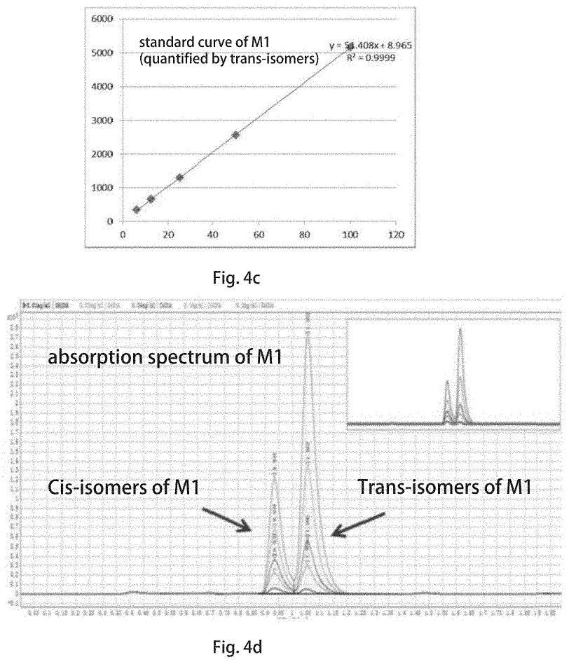

The curcumin analog M1 prepared in Embodiment 1 is dissolved in acetonitrile, and the acetonitrile solution of the curcumin analog M1 having concentration series (6.25, 12.5, 25, 50, and 100 μg/mL) are configured in gradients, and light absorbance is measured at 428 nm using ultra-high performance liquid chromatography (UPLC) to make a standard curve. Three groups of 100 μg/mL of the nanoparticles of the curcumin analog M1 are taken and respectively dissolved in acetonitrile, processed under ultrasonic sound for 5 minutes, and evaluated in the same method. A curcumin analog M1 content of the nanoparticles of the curcumin analog M1 is calculated based on the standard curve. Referring to FIG. 4a, a drug load rate of the nanoparticles of the curcumin analog M1 is (31.49±2.03)%.

### (3) Evaluation of Surface Potential of the Nanoparticles of the Curcumin Analog M1

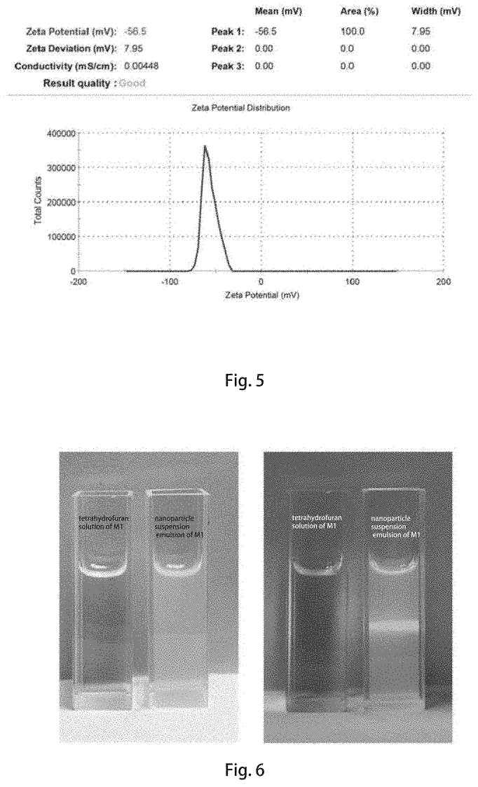

The laser particle size analyzer is used according to the method described in the specification of the laser particle size analyzer to analyze Zeta-potential of the nanoparticles of the curcumin analog M1 prepared in Embodiment 1. An average charge of the nanoparticles of the curcumin analog M1 prepared in Embodiment 1 is −56.5 mV, and FIG. 5 illustrates a distribution of the surface potential.

### (4) Evaluation of Optical Characters of the Nanoparticles of the Curcumin Analog M1

Referring to FIG. 6, small molecules of the curcumin analog M1 prepared in Embodiment 1 and the nanoparticles of the curcumin analog M1 prepared in Embodiment 2 are respectively dissolved in water and organic solvents. The small molecules of the curcumin analog M1 are difficultly dissolved in water while are soluble in tetrahydrofuran. However, the nanoparticles of the curcumin analog M1 can be dispersed in water and have Tyndall effect under laser light.

### (5) Evaluation of a Drug Release Curve of the Nanoparticles of the Curcumin Analog M1

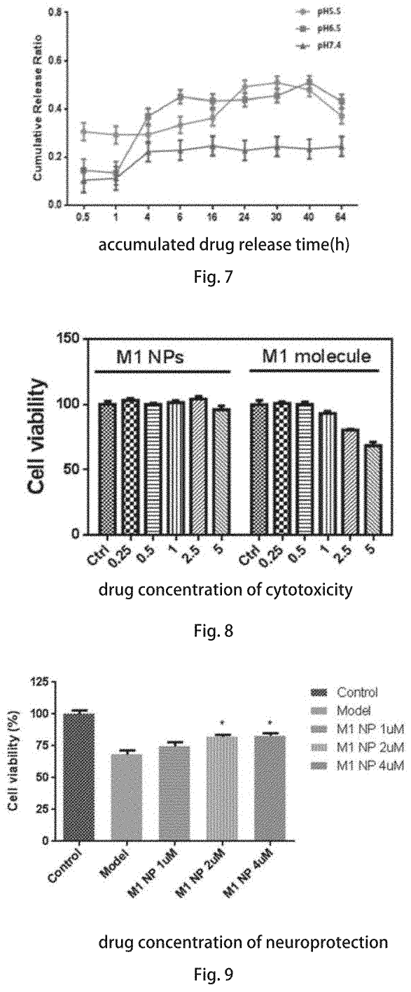

The nanoparticles of the curcumin analog M1 prepared in Embodiment 2 are equally divided into six parts, 3 parts are added into artificial nasal fluid, 3 parts are added into 5% plasma, dispersed, diluted, and respectively added into dialysis bags (3500 molecular weight, Fisherbrand, U.S.), then immersed in a 200 mL buffer having the same pH, and continuously stirred at 37° C. 2 mL solution is collected from the solution (i.e., the buffer) at a preset time point. During the dialysis process, 2 mL phosphate-buffered saline (PBS) is supplemented after each sampling to keep a volume of the solution to be constant. Light absorbance is evaluated by an ultraviolet-visible spectroscopy (UV-VIS) method, and an amount of drug release is calculated. Each sample is tested 3 times. An average value is taken to perform statistical analysis, and FIG. 7 illustrates the results. The nanoparticles of the curcumin analog M1 prepared in Embodiment 2 have slow release properties and release slowly and stably instead of having an initial explosive drug release. It is important for applying the nanoparticles of the curcumin analog M1 in vivo, drug toxicity is reduced, and drug leakage is reduced, etc.

### (6) Cytotoxicity Test

Neuroma blast cells N2a are cultured according to a method described in the literature (Cell Culture, Zhenqiang SITU, World Publishing Corporation, 1996), and then the nanoparticles of the curcumin analog M1 prepared in Embodiment 2 are added to be continually cultured. A cell survival rate is evaluated according to a method (i.e., Thiazolyl Blue Tetrazolium Bromide (MTT) method) described in the literature (Cell Culture, Zhenqiang SITU, World Publishing Corporation, 1996) 24 hours after drug addition, which defines a group of the nanoparticles of the curcumin analog M1. A group of Neuroma blast cells N2a that are treated with free curcumin analog M1 having the same concentration as the group of the nanoparticles of the curcumin analog M1 by the same method defines positive control group. A group of Neuroma blast cells N2a that are cultured in a blank medium without hydrophobic drugs (i.e., the curcumin analog M1) defines a negative control group, and a rate of cell viability of the Neuroma blast cells N2a in the negative control group is calculated as 100%. FIG. 8 illustrates the results. As a concentration becomes higher, the free curcumin analog M1 show dose-dependent cytotoxicity. However, the nanoparticles of the curcumin analog M1 prepared in Embodiment 2 have no cytotoxic effect on the Neuroma blast cells N2a at the same concentrations. It is possible that an accumulation toxicity of a small molecule drug of the curcumin analog M1 at higher concentrations is inhibited due to a slow release effect of the nanoparticles of the curcumin analog M1.

### (7) Evaluation of the Neuroprotective Effect of the Nanoparticles of the Curcumin Analog M1

Nerve cell lines PC12 are processed to obtain a neurotoxic cell model using neurotoxin 1-methyl-4-phenylpyridinium (MPP⁺). The nanoparticles of the curcumin analog M1 prepared in Embodiment 2 are added for pre-treating 6 hours before the modeling to define a group of the nanoparticles of the curcumin analog M1. No drug is added to define a model control group, and no neurotoxin MPP⁺ is added to define a normal control group. After the modeling, continually culturing for 48 hours, and light absorbance is evaluated according to the method described in the literature. FIG. 9 illustrates the results. A cell viability rate of the group of the nanoparticles of the curcumin analog M1 is significantly higher than that of the MPP⁺ model control group. The nanoparticles of the curcumin analog M1 prepared in Embodiment 2 dose-dependently protect the nerve cell lines PC12 and reduce a cell damage induced by neurotoxin MPP⁺.

### (8) Evaluation of a Binding Effect of the Nanoparticles of the Curcumin Analog M1 in Combination with a TFEB Protein of a Target Protein

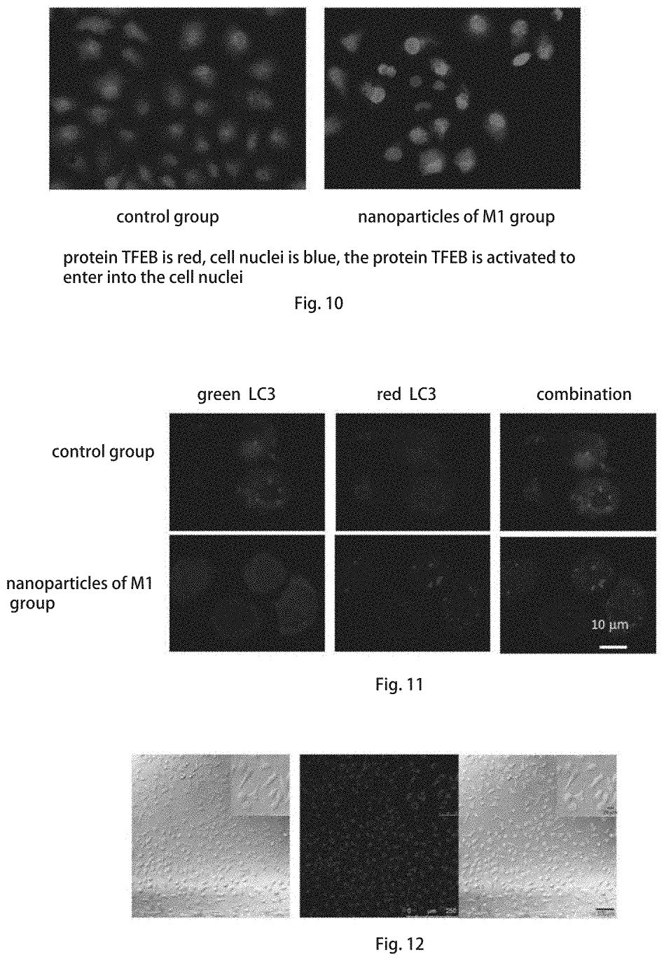

The target protein of molecules of the free curcumin analog M1 having neuroprotective effect is the TFEB protein in cytoplasm. The curcumin analog M1 promotes the TFEB protein to be dephosphorylated to enter into cell nuclei, and an expression of autophagy related genes increases, thereby providing a neuroprotective effect. In this experiment, the nanoparticles of the curcumin analog M1 prepared in Embodiment 2 are added to MF7 cells with the overexpressed protein TFEB that is fluorescently labeled. After 24 hours of treatment, a state at which the protein TFEB enters into the nuclei is observed, and FIG. 10 illustrates the results. The nanoparticles of the curcumin analog M1 prepared in Embodiment 2 promote the protein TFEB to dose-dependently enter into the nuclei, and which confirms that nanoparticles of the curcumin analog M1 retain targeting characters of original molecules.

### (9) Induction of the Nanoparticles of the Curcumin Analog M1 on Cell Autophagy Flux

When autophagy is induced, an expression of marker protein LC3 increases. A lentivirus expressing GFP-RFP-LC3 is constructed and infects the Neuroma blast cells N2a, and drug effects on autophagy flux can be evaluated under a confocal fluorescence microscope. Protein GFP-LC3 is a green acid-responsive protein that can be degraded in acid lysosomes, while protein RFP-LC3 is a red acid-stable protein that will not be degraded in lysosomes. Therefore, when autophagy pathway is activated and the autophagy flux is smooth, red protein RFP-LC3 spots increase, which indicates an induction of the autophagy flux. In the Neuroma blast cells N2a infected with the lentivirus, the nanoparticles of the curcumin analog M1 prepared in Embodiment 2 are added for drug treatment, which defines a group of the nanoparticles of the curcumin analog M1. After 24 hours, a number of the red protein RFP-LC3 spots is evaluated under a confocal microscope. Referring to FIG. 11, compared with a control group, the number of the red protein RFP-LC3 spots in cells of the group of the nanoparticles of the curcumin analog M1 significantly increase, which confirms that the nanoparticles of the curcumin analog M1 can induce an occurrence of the autophagy flux. This is a knock-on effect after the protein TFEB is activated in the results of the test (8).

### Embodiment 3: a Brain-Targeted Delivery System of the Intranasal Nano-Formulation of the Curcumin Analog M1 Loading Fluorescent Probe TPAAQ (e.g., the Nanoparticles of the Curcumin Analog M1 Loading Fluorescent Probe TPAAQ)

TPAAQ is a small hydrophobic molecule fluorescent probe that is excited at 473 nm and emitted at 650 nm, and it can be used to monitor a fluorescence distribution in vivo of nanomaterials. As it is also a small hydrophobic molecule, like a preparation process of the nanoparticles of the M1 prepared in Embodiment 1, the self-contained intranasal nano-formulation of the curcumin analog M1 loading fluorescent probe TPAAQ without carriers can be prepared in the same way.

5 mL of a tetrahydrofuran solution containing 1 mg/mL of the curcumin analog M1 and 2 mg/mL of TPAAQ is configured and well mixed, 200 μL of the curcumin analog M1 solution is taken and added dropwise to 5 mL of deionized water, and nitrogen gas is blown while dripping to remove organic solvents. Magnetically stirring at 25° C. for 10 minutes, then still standing to obtain a suspension emulsion of self-contained nanoparticles of the curcumin analog M1 loading the fluorescent probe TPAAQ without carriers, and then freeze-drying to obtain a freeze-dried powder. Before immediate use, the freeze-dried powder is redispersed in isotonic saline, added dropwise into a chitooligosaccharide saline solution (0.1 w/v), stirred for 0.5-2 hours, reacted for 1 hour by physical adsorption, and centrifuged at a speed of 10000-150000*g for 5-30 minutes. A supernatant is discarded, reactants are removed to purify the product, and the self-contained intranasal nano-formulation loading fluorescent probe TPAAQ without carriers that is modified by chitooligosaccharides is obtained.

### Results

### (1) Evaluation of Morphology, Size Distribution of the Nanoparticles of the Curcumin Analog M1 Loading the Fluorescent Probe TPAAQ

A scanning electron microscope (FEI Quanta200, Netherlands) is used to observe the nanoparticles of the curcumin analog M1 prepared in Embodiment 2 according to a method described in a specification of the scanning electron microscope. FIG. 1b illustrates a scanning electron micrograph. A high-resolution transmission electron microscope (FEI Technai F30, Netherlands) is used to observe the nanoparticles of the curcumin analog M1 prepared in Embodiment 2 according to a method described in a specification of the high-resolution transmission electron microscope. FIG. 2b illustrates a transmission electron micrograph. A laser particle size analyzer (Malvern, UK) is used to evaluate dynamic light scattering of the nanoparticles of the curcumin analog M1 prepared in Embodiment 2 according to a method described in a specification of the laser particle size analyzer. An average particle size of the nanoparticles of the curcumin analog M1 prepared in Embodiment 2 is evaluated to be 178.2 nm, and FIG. 3b illustrates a particle size distribution.

### (2) Evaluation of a Drug Load Rate of the Nanoparticles of the Curcumin Analog M1 Loading the Fluorescent Probe TPAAQ

The standard curve of the acetonitrile solution and the curcumin analog M1 prepared in the test (2) of Embodiment 1 is used. Three groups of 100 μg/mL of the nanoparticles of the curcumin analog M1 loading the fluorescent probe TPAAQ are taken and respectively dissolved in acetonitrile, processed under ultrasonic sound for 5 minutes, and evaluated in the same method. A curcumin analog M1 content of the nanoparticles of the curcumin analog M1 is calculated based on the standard curve. Referring to FIG. 4b, a drug load rate of the nanoparticles of the curcumin analog M1 loading the fluorescent probe TPAAQ is (26.95±1.50)%.

### (3) Cell Uptake Test

Nerve cells are normally cultured, and the intranasal nano-formulation of the curcumin analog M1 loading the fluorescent probe TPAAQ prepared in Embodiment 3 are added. After culturing for 3 hours, the cell uptake is observed with a laser confocal scanning microscope at a preset wavelength. Referring to FIG. 12, a large amount of the intranasal nano-formulation of the curcumin analog M1 loading the fluorescent probe TPAAQ prepared in Embodiment 3 can be up-taken by cells according to fluorescence signals.

### Embodiment 4: Application of a Nasal-Brain Targeted Delivery System of the Nanoparticles of the Curcumin Analog M1

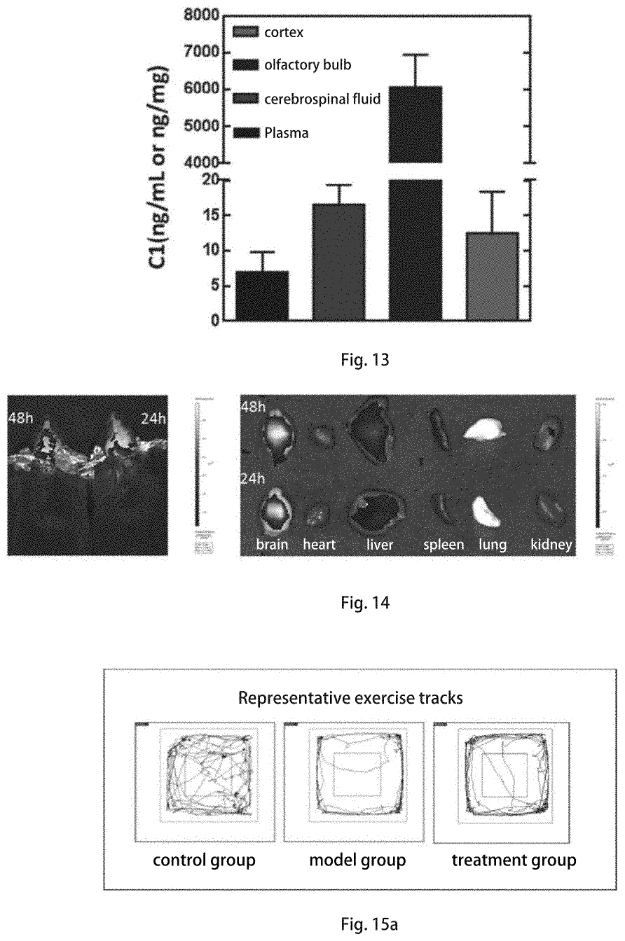

Six male mice of C57BL/6J strain with a body weight of 25 g are taken and adaptively reared for 3 days. The intranasal nano-formulation of the curcumin analog M1 prepared in Embodiment 2 is dispersed in isotonic saline. A concentration of the intranasal nano-formulation of the curcumin analog M1 is 5 mg/mL, and 15 μL is administered to a nasal cavity of a mouse. After 24 hours, brain tissues, cerebrospinal fluid, and plasma are dissected and taken out. The brain tissues are divided into olfactory bulbs and the rest of a brain. All samples are added with methanol to remove proteins, and then a triple quadrupole HPLC-MS is used to analyze a drug content of the curcumin analog M1 in the samples. FIG. 13 illustrates the results. A brain-targeted delivery system of the intranasal nano-formulation of the curcumin analog M1 highly and selectively delivers the curcumin analog M1 into the olfactory bulb, and a distribution in the cerebrospinal fluid is three times higher than that in the plasma. A distribution in the rest of the brain is twice the amount of plasma. It confirms that an absorption pathway of the curcumin analog M1 to the brain extends through the olfactory bulb, and the curcumin analog M1 can be transmitted to the rest of the brain. A transmission of the curcumin analog M1 may be time-dependent, and it will continue to be subsequently transmitted from the cerebrospinal fluid after 24 hours.

### Embodiment 5: Application of the Nanoparticles of the Curcumin Analog M1 Loading the Fluorescent Probe TPAAQ Using the Nasal-Brain Targeted Delivery System

Nine male mice of the C57BL/6J strain with a body weight of 25 g are taken and adaptively reared for 3 days. The intranasal nano-formulation of the curcumin analog M1 loading the fluorescent probe TPAAQ prepared in Embodiment 3 is dispersed in saline. A concentration of the curcumin analog M1 is 5 mg/mL, and 15 μL is administrated to a nasal cavity of a mouse. After 24 hours and 48 hours, respectively, a small animal fluorescence imaging system is used to evaluate fluorescence signals in the brain of the mice in vivo, as well as fluorescence signals in organs and blood in vitro. The organs can be the brain, a heart, a liver, a spleen, a lung, a kidney, etc. FIG. 14 illustrates the results. The fluorescence signals of the brain are significantly stronger than other parts and tissues of a body, which suggests that brain targeted delivery system of Embodiment 3 can successfully highly and selectively deliver the intranasal nano-formulation of the curcumin analog M1 into the brain, and a distribution of the drug in peripheral tissues is reduced.

### Embodiment 6: Therapeutic Application of the Self-Contained Intranasal Nano-Formulation of the Curcumin Analog M1 Without the Carriers in Parkinson Model Mice

Thirty male mice of the C57BL/6J strain with a body weight of 25 g are taken and divided into three groups. A first group is a wild-type group (WT group), a second group is a model group (MPTP group), and a third group is a model administration group (M1 NPs), 10 mice per group. According to the method described in the literature, mice of the second group and the third group are continuously and intraperitoneally injected with 1-methyl-4-phenyl-1,2,3,6-tetrahydropyridine (MPTP) neurological toxin at a dose of 20 mg/kg for 5 days to create Parkinson's disease models. Mice of the WT group and MPTP group are administrated saline through the nasal cavity, and the M1 NPs group is administrated the self-contained intranasal nano-formulation of the curcumin analog M1 without the carriers. That is, the intranasal nano-formulation of the curcumin analog M1 prepared in Embodiment 2 is dispersed in isotonic saline, and a new formulation is prepared for immediate use. A concentration of the curcumin analog M1 is 1 mg/mL, and 15 μL are administrated to the mice through the nasal cavity. The drug is administered every two days, four times in total, and behaviors are observed two weeks after a completion of the modeling. The mice are then dissected, the brain tissues are separated, and various pharmacological tests are performed.

### Results

### (1) Behavioral Performance of Parkinson's Model Mice is Evaluated by an Open Field Test

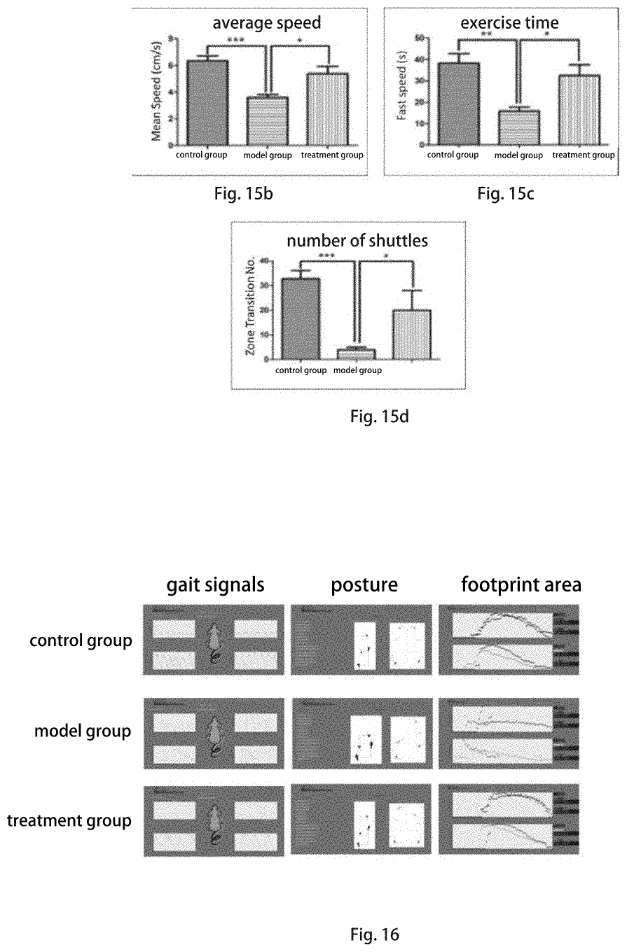

The MPTP Parkinson's mice model has symptoms such as movement disorders and significant anxiety, which can be detected by the open field test. According to the method described in the literature, the behavioral performance of Parkinson's model mice in Embodiment 5 is tested. FIG. 15a illustrates the results. Compared with the wild-type mice in the control group, movement tracks of the mice of the model group change significantly, and after treatment of the intranasal nano-formulation of the curcumin analog M1 , the movement tracks tends to be normal. Statistics show that compared with wild-type mice, exercise time (FIG. 15c), average speed (FIG. 15b), the number of shuttles (FIG. 15d), etc. of model mice in an open filed are significantly reduced. After treatment of the self-contained intranasal nano-formulation of the curcumin analog M1 without the carriers, the above-mentioned pathological changes are significantly improved, which confirms that the intranasal nano-formulation of the curcumin analog M1 can effectively alleviate behavioral symptoms of Parkinson's disease.

### (2) The Behavioral Performance of the Parkinson Model Mice is Evaluated by Gait Test

Clinical symptoms of Parkinson's disease mainly include resting tremor, bradykinesia, muscle rigidity, postural disorder, and gait disorder. A DigiGait imaging system is used on animals. Animals are imagined under a transparent running belt. Software is used to quantify features, such as gait mechanics and posture index, to detect behavioral characters of the Parkinson's model mice. FIG. 16 illustrates the results. Compared with the wild-type mice, the Parkinson's model mice have disordered gait signals, decreased coordination, and significantly reduced footprint area. After treatment with the self-contained intranasal nano-formulation of the curcumin analog M1 without the carriers, the above-mentioned pathological changes are significantly improved, which confirms that the intranasal nano-formulation of the curcumin analog M1 can effectively improve behavioral symptoms of Parkinson's disease.

### (3) A Distribution of the Olfactory Bulb, Striatum, and Substantia Nigra After Administration of the Nanoparticles of the Curcumin Analog M1 from the Nasal Cavity of the Parkinson'S Model Mice is Evaluated Using Tissue Electron Microscopy

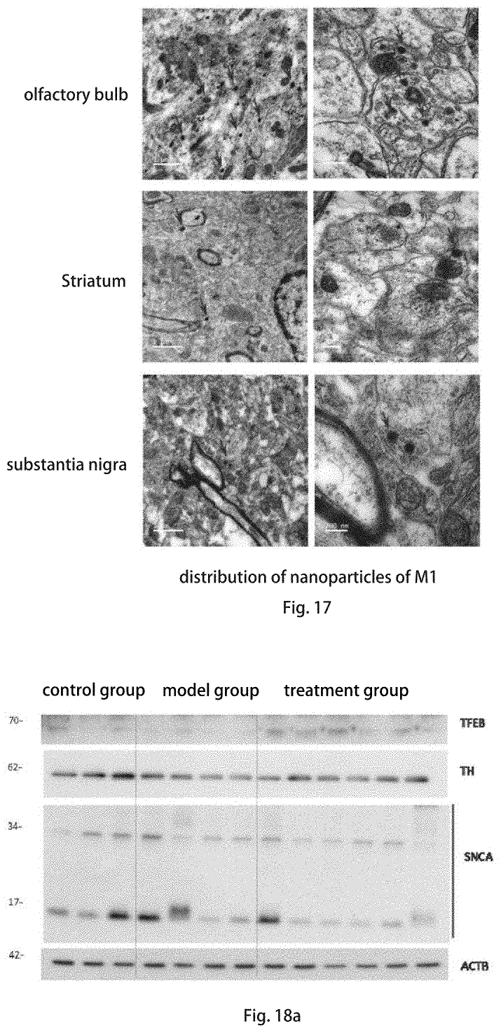

Three mice in the treatment group are taken and dissected 24 hours after the last nasal administration of the intranasal nano-formulation of the curcumin analog M1. The brain tissue is taken out, the olfactory bulb, the striatum, and the substantia nigra are separated, sections are fixed, and a distribution of various areas of the brain of the nanoparticles of the curcumin analog M1 are observed under a transmission electron microscope. Referring to FIG. 17, it is clearly observed that the nanoparticles of the curcumin analog M1 are distributed in the olfactory bulb, the striatum, and the substantia nigra of the brain. It confirms that after intranasal administration of the intranasal nano-formulation of the curcumin analog M1, prototypes of the nanoparticles of the curcumin analog M1 can enter tissues of the brain, and a distribution of the olfactory bulb is the largest, which suggests that its absorption pathway is mediated by the olfactory nerve and subsequently diffuses to other areas.

### Expression Levels of Toxic Proteins and Autophagy Pathway-Related Proteins in the Olfactory Bulb of the Brain are Evaluated Using a Western Blot Method

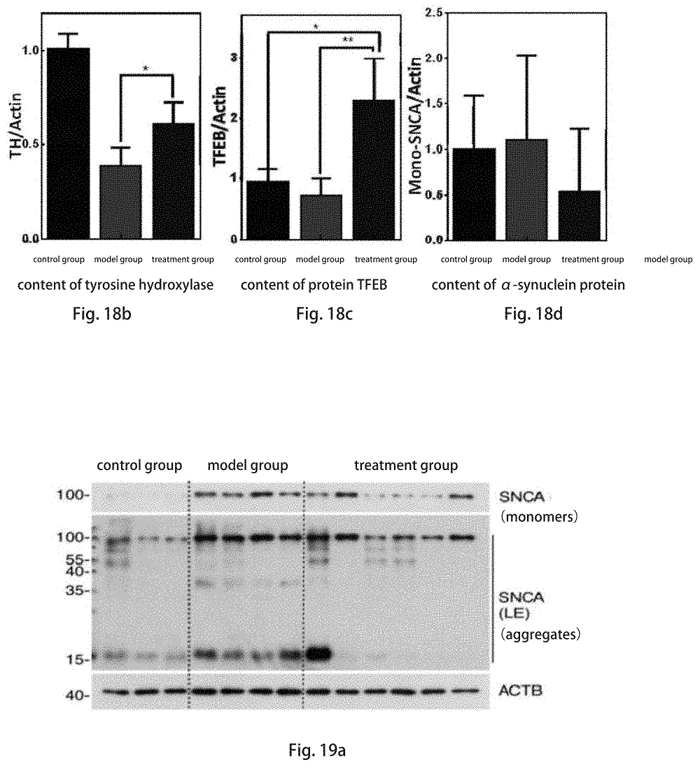

Tyrosine hydroxylase (TH) is a key enzyme in a dopamine biosynthesis pathway, protein synuclein alpha (SNCA) is an α-synuclein protein that accumulates in the brain of patients suffering from Parkinson's disease, and the protein TFEB (e.g., the toxic protein TFEB) is a protein related to the autophagy pathway. After the administration in the mice in each group is complete, the mice in the control group, the model group, and the treatment group are sacrificed. The brain tissues are taken, the olfactory bulb is separated, and a total amount of protein is evaluated after a tissue homogenization. According to conventional steps of the Western Blot method, antibodies of the above-mentioned protein of Santa Cruz company are used to detect expression levels of the above-mentioned proteins in the homogenate of the olfactory bulb. FIG. 18a illustrates the results. Statistics indicate that tyrosine hydroxylase (TH) protein is significantly reduced in the model group while significantly increased (FIG. 17) in the treatment group, which confirms that the intranasal nano-formulation of the curcumin analog M1 can alleviate toxin-induced dopamine synthesis disorder in the brain. Compared with the control group and the model group, an amount of the protein TFEB in the olfactory bulb of the brain of the treatment group is significantly increased (FIG. 18c), which confirms that the intranasal nano-formulation of the curcumin analog M1 activates the protein TFEB in the olfactory bulb, which is one of possible mechanism for drug effects. In addition, a content of the toxic protein SNCA is increased in the model group while the content of the toxic protein SNCA has a decreasing trend (FIG. 18d) in the treatment group, which confirms that the intranasal nano-formulation of the curcumin analog M1 can eliminate the toxic protein in the olfactory bulb. Therefore, after nasal administration of the intranasal nano-formulation of the curcumin analog M1, an accumulation of neurotoxins in the olfactory bulb can be eliminated, and the dopamine synthesis disorders caused by the toxins can be alleviated. The drug effects may be related to that the protein TFEB -mediated autophagy pathway is induced by the curcumin analog M1.

### (5) Expression Levels of Toxic Proteins and Autophagy Pathway Related Proteins in the Substantia Nigra of the Brain are Evaluated Using the Western Blot Method

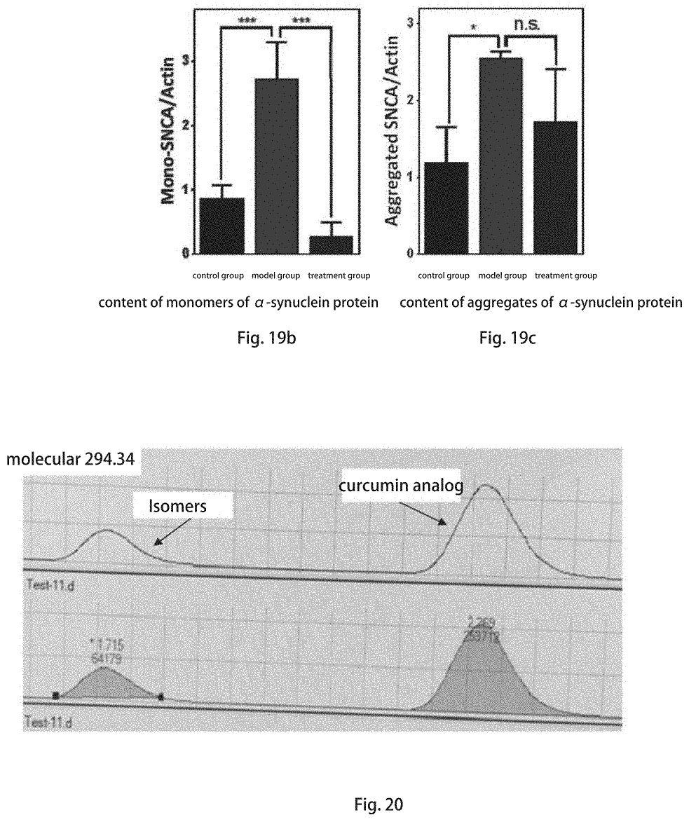

After the administration in the mice in each group is complete, the mice in the control group, the model group, and the treatment group are sacrificed. The brain tissues are taken, the substantia nigra is separated, and a total amount of protein is evaluated after tissue homogenization. According to the conventional steps of the Western Blot method, α-synuclein protein antibody of Santa Cruz company is used to evaluate an expression of monomers of α-synuclein protein and aggregates of α-synuclein protein in the homogenate of the substantia nigra. FIG. 19a illustrate the results. Statistics indicate that the monomers and the aggregates of the α-synuclein protein are significantly increased in the model group while the monomers and the aggregates of the α-synuclein protein have a significant decreasing trend in the treatment group (FIGS. 19b and 19c), which confirms that the intranasal nano-formulation of the curcumin analog M1 can effectively remove an accumulation of toxic proteins in lesions of the substantia nigra, thereby playing a therapeutic effect on the disease.

The aforementioned embodiments are used to describe detailed technical features and detailed method of the present disclosure, and the scope of the disclosure is not limited thereto. That is, the present disclosure cannot be achieved only based on the aforementioned detailed technical features and detailed method. Thus, it is intended that the present disclosure cover any improvements of the present disclosure, equivalent replacements of selected composition, addition of auxiliary ingredients, and selection of a detailed method for those skilled in the art.

What is claimed is:

1. An inducer, comprising:
cis-isomers or a mixture of cis-isomers and trans-isomers of a hydrophobic curcumin analog, wherein:
the cis-isomers or the mixture of cis-isomers and trans-isomers of the hydrophobic curcumin analog has the following structural formula generated by exposure to sunlight, ultraviolet, or radioactive radiation:

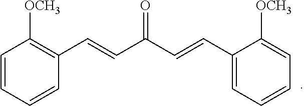 [SMILES: COc1ccccc1/C=C/C(=O)/C=C/c1ccccc1OC]

2. The inducer according to claim 1, wherein a weight ratio of the cis-isomers in the mixture is 25-35% of the total mixture.

3. The inducer according to claim 1, wherein:
a surface of the inducer is decorated with surfactant that has a lipophilic group and a hydrophilic group, and is configured to be used in combination with the inducer to form self-assembled nanoparticles.

4. The inducer according to claim 1, wherein the inducer is used for a treatment of neurodegenerative diseases comprising Alzheimer's disease and Parkinson's disease.

5. The inducer of claim 4, wherein the neurodegenerative diseases are accompanied with olfactory disorder symptoms, and the inducer is an intranasal nano inducer that is targeted and enriched in olfactory bulb.

6. An intranasal nano inducer for preventing and treating neurodegenerative diseases, wherein:
the intranasal nano inducer is the inducer according to claim 1, and
a dosage formulation is a nasal spray or nasal drops.

7. A method for preparing an intranasal nano inducer for preventing and treating neurodegenerative diseases, comprising:
configuring a solvent solution having 1-10 mg/mL of amphiphilic surfactant and 0.5-5 mg/mL of hydrophobic molecules having inducing effects,
then dripping the solvent solution into deionized water, wherein a volume ratio of the solvent solution and the deionized water is (0.5-5):50,
blowing gas during the dripping to assist in a volatilization of a solvent of the solvent solution,
preparing a suspension emulsion of self-contained nanoparticles without carriers with a particle size of 50-200 nm by a reprecipitation method,
freeze-drying to prepare a freeze-dried powder, and
resuspending the freeze-dried powder in isotonic saline to obtain the intranasal nano inducer before use.

8. The method for preparing the intranasal nano inducer for preventing and treating neurodegenerative diseases according to claim 7, wherein:
the hydrophobic molecules having inducing effects are a curcumin analog of the following structural formula, cis-isomers of the curcumin analog, or a mixture of cis-isomers and trans-isomers in any ratio of the curcumin analog:

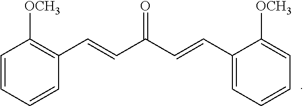 [SMILES: COc1ccccc1/C=C/C(=O)/C=C/c1ccccc1OC]

9. The method for preparing the intranasal nano inducer for preventing and treating neurodegenerative diseases according to claim 8, comprising wherein preparing the mixture comprises irradiating a methanol solution of the curcumin analog with ultraviolet radiation for 1.5-2.5 hours, wherein a weight ratio of the cis-isomers in the mixture is 25-35% of the total mixture.

10. The method for preparing the intranasal nano inducer for preventing and treating neurodegenerative diseases according to claim 7, wherein a concentration of chitooligosaccharides in the isotonic saline is 0.01-0.2% (w/v).

11. The method for preparing the intranasal nano inducer for preventing and treating neurodegenerative diseases according to claim 7, wherein:
the amphiphilic surfactant is a polyethylene glycol derivative, and
the polyethylene glycol derivative is a negatively charged polyethylene glycol derivative.

12. The method for preparing the intranasal nano inducer for preventing and treating neurodegenerative diseases according to claim 11, wherein the polyethylene glycol derivative is a polyethylene glycol derivative having a carboxyl group or polymaleic anhydride 18 carbene-polyethylene glycol.

13. A method for preparing a intranasal nano inducer for preventing and treating neurodegenerative diseases, comprising:
configuring a solvent solution having 1-10 mg/mL of polyethylene glycol derivative and 0.5-5 mg/mL of hydrophobic molecules,
then dripping the solvent solution into deionized water, wherein a volume ratio of the solvent solution to the deionized water is (0.5-5):50,
blowing gas during the dripping to assist in a volatilization of a solvent of the solvent solution,
preparing a suspension emulsion of self-contained nanoparticles without carriers having a particle size of 50-200 nm using a reprecipitation method,
freeze-drying to prepare a freeze-dried powder, and
resuspending the freeze-dried powder in isotonic saline to obtain the intranasal nano inducer before use.

14. An intranasal nano inducer for preventing and treating neurodegenerative diseases, wherein:
the intranasal nano inducer is the inducer according to claim 2, and
a dosage formulation is a nasal spray or nasal drops.

15. An intranasal nano inducer for preventing and treating neurodegenerative diseases, wherein:
the intranasal nano inducer is the inducer according to claim 3, and
a dosage formulation is a nasal spray or nasal drops.

16. An intranasal nano inducer for preventing and treating neurodegenerative diseases, wherein:
the intranasal nano inducer is the inducer according to claim 4, and
a dosage formulation is a nasal spray or nasal drops.

17. The method for preparing the intranasal nano inducer for preventing and treating neurodegenerative diseases according to claim 8, wherein a concentration of chitooligosaccharides in the isotonic saline is 0.01-0.2% (w/v).

18. The method for preparing the intranasal nano inducer for preventing and treating neurodegenerative diseases according to claim 8, wherein:
the amphiphilic surfactant is a polyethylene glycol derivative, and
the polyethylene glycol derivative is a negatively charged polyethylene glycol derivative.

19. The inducer according to claim 3, wherein the inducer is used for a treatment of neurodegenerative diseases comprising Alzheimer's disease and Parkinson's disease.

20. The method for preparing the intranasal nano inducer for preventing and treating neurodegenerative diseases according to claim 7, wherein the hydrophobic molecules having inducing effects are a curcumin analog.
